(19) United States
(12) Patent Application Publication
(10) Pub. No.: US 2008/0226503 A1
(43) Pub. Date: Sep. 18, 2008

(54) Method for selective detection of peptides using molecularly imprinted sensors

(76) Inventors: Dar-Fu Tai, Hualien (TW); Chung-Yin Lin, Hualien (TW); Tzong-Zeng Wu, Hualien (TW)

Correspondence Address: DAR-FU TAI, 937 HELENA CIR, COSTA MESA, CA, 92626, US

(21) Appl. No.: 12/109,352

(22) Filed: Apr. 25, 2008

Related U.S. Application Data
(63) Continuation-in-part of application No. 10/690,600, filed on Oct. 23, 2003, now abandoned.

(51) Int. Cl. G01N 33/48

(52) U.S. Cl. 422/68.1

(57) ABSTRACT

This invention presents a fast and effective method to detect macromolecules such as peptides. Using a multifunctional chiral monomer, it combines molecular imprinting polymerization technology with a quartz crystal microbalance for detection of peptides to the ng/ml scale.

### CROSS REFERENCE

This application is a continuation-in-part (CIP) of application Ser. No. 10/690,600, filed on Oct. 23, 2003. The prior application is herewith incorporated by reference in its entirety.

### BACKGROUND OF THE INVENTION

A quick and simple way to identify analytes in clinical diagnosis and environmental analysis is desirable. Quartz crystal microbalance (QCM) is a bulk-acoustic wave resonator. It determines the change of mass by measuring a change in frequency. QCM is widely used in sensors because of its simplicity, sensitivity and instantaneous reading. What is lacking is selectivity.

Molecularly imprinted polymerization is a process of synthesizing polymers with cavities of template molecules. These cavities provide a degree of recognition through steric and electrostatic interactions. Molecularly imprinted polymers (MIPs) often show a high selectivity towards their substrate similar to antibody-antigen complementarities. As a synthetic compound, it provides a good alternative to biological receptors because of its stability. Typically, MIPs are prepared in three steps: 1) mixing template molecules with one or more functional monomers in a solvent; 2) polymerization of template-monomer complex in the presence of a cross-linker; and 3) removal of the template molecules from imprinted polymers to yield polymers with recognition cavities. Factors such as concentration of cavities, physical shape, distribution, rigidness and depth of cavities have great impact on the selectivity of MIPs.

Currently, the majority of MIP preparation is based upon noncovalent interactions of template molecules and functional monomers. In general, MIPs prepared with this method suffer poor specificity/selectivity due to relatively weak noncovalent interactions. In addition, reagents used in polymerization and/or copolymerization may sometimes cause undesired effect and reduce the quality of MIPs produced. For example, initiators used in activation process often limit density of MIPs, Claudia Sulitzky et al.; Macromolecules 2002, 35, 79-91, incorporated herein by reference. Thus, choosing an appropriate monomer has always been a major challenge for MIP fabrication.

Using conventional MIP preparation protocol, Kempe M.; Letters in Peptide Science, 7: 27-33, 2000, incorporated herein by reference, had prepared Z-oxytocin imprinted MIPs by polymerizing a mixture of Z-oxytocin, methacrylic acid, cross-linker and azo-N,N′-bis-isobutyronitrile (AIBN). The resulting polymer was ground and wet-sieved. Because oxytocin was not used as the template molecule, oxytocin detection can only be observed at high concentration.

Approaches to increase MIP selectivity have focused on improving functional monomers. U.S. Pat. No. 6,890,486, incorporated herein by reference, describes a MIP/QCM sensor where an enhanced crosslinking monomer was used to fabricate non-SAM (self-assembled monolayer) MIPs for detection of environmental contaminants.

Still, the lack of highly selective MIPs to some group of compounds necessitates the search for new solutions. This invention presents a MIP/QCM sensor that can selectively detect biomolecules, such as peptides and amino acid, at very low concentration. It significantly boosts the specificity/selectivity of MIPs through a new functional monomer capable of strengthening binding to compounds with hydrophobic groups such as benzyls, phenyls, phenols and, most importantly, enhancing cavity affinity through various aspects.

### SUMMARY OF THE INVENTION

The objective of this invention is to provide a method of building a highly selective and sensitive quartz crystal microbalance (QCM) sensor by coating a molecularly imprinted polymer (MIP) film on quartz crystal to screen for specified macromolecules.

First, a synthesized multi-functional chiral monomer was chemically coated onto a metal chip to form a chiral self-assembled monolayer (SAM) film. Then a liquid mixture of chosen template molecules and monomers was prepared. The mixture was polymerized to form a chiral SAM MIP coating on the chip. As the last step of preparation, template molecules were extracted with solvent to render a MIP/QCM chip with recognition cavities. The chip was immersed in a flow injection system connected to a data processing machine to record frequency change as test samples were injected.

This detection method is particularly useful for achieving good sensitivity and selectivity in low concentration of peptides.

In summary, this invention introduces a specialized chiral reagent; combining with MIP and QCM technologies, it provides a fast and qualitative as well as quantitative way of detecting specified biomolecules.

### BRIEF DESCRIPTION OF DRAWINGS

NONE

### DETAILED DESCRIPTION OF THE INVENTION

Here we disclose the method of fabricating a highly selective MIP onto a QCM chip, and subsequently demonstrate the sensor's response by measuring the frequency change. Detail of this invention is described in three steps: choosing monomers, preparation of MIPs with template molecules, and evaluation of MIP-grafted QCM.

### (1) Choosing Monomers

As mentioned earlier, the quality of MIPs is greatly influenced by the geometry, concentration and distribution of recognition cavities. With this in mind, a self-assembled monomer with chirality was developed to construct a SAM coating on the surface of a quartz crystal and serve as the foundation of MIP building block. This monomer is multi-functional as it is also used as a cross-linker and a photoinitiator. These features, together with its chiral structure, afford a MIP with cavities of exceptional affinity, selectivity and sensitivity.

This compound is a derivative of cystine, such as L-cystine, D-cystine, L-homocystine, and D-homocystine. Here we use the term cystine-derivative to refer to such cystine derivative in the rest of this application. The structure of a sample cystine-derivative, bisacryloyl-L-cystine-bis-benzylamide, synthesized for this invention is shown in the figure below. Here we use (Acr-L-Cys-NHBn)₂ to refer to formula of this compound.

The cystine-derivative contains two chiral centers. With the chiral center's 3-dimensional character and SAM's lateral distribution feature, a chiral SAM MIP film with significant specificity can be obtained. Furthermore, SAM character results in a secure and firm binding between the compound and the metal surface. The aromatic rings on the said compound will introduce a hydrophobic surface on MIPs after polymerization and it increases the affinity of MIPs. To further enhance MIP affinity, another hydrophobic monomer, N-benzyl-acrylamide is used in copolymerization.

Unlike typical alkylthiol acrylic monomers that are symmetric and provide only a single-point attachment to a surface, cystine-derivatives also act as a cross-linker. It forms a stable and rigid foundation between MIPs and metal electrode surface after polymerization. This yields a MIP with additional affinity and selectivity.

Furthermore, cystine-derivatives will generate a free radical group upon irradiation: this allows polymerization without an additional photoiniator and produces polymers in solution. This is ideal, as regular photoiniating agents may form undesired, nonspecific cavities on the surface as well as free polymers in the solution reducing the quality of MIP.

The sample (Acr-L-Cys-NHBn)₂ was synthesized from N,N′-diBoc-L-cystine ((BOC-L-Cys)₂). Those skilled in the art would be able to obtain a similar compound using suitable synthesis reagents. The figure below illustrated preparation of the above compound afforded in 50% yield.

Alternatively, chiral derivatives of cystine and homocystine not only having benzyl or phenyl but also containing acryloyl or methacryl can be used in place of (Acr-L-Cys-NHBn)₂. A sample bismethacryl-L-cystine-bis-benzylamide has been synthesized and afforded a 30% yield.

(2) Preparation of MIP with Template Molecules

Template molecules considered useful for this invention are disulfide bonded cyclic peptides including, but not limited to, those with functional groups such as cysteine, cystine, phenol, benzene, imidazole, guanidinium, alcohol, disulfide, thiol, or amide.

We hereby use examples below to demonstrate preparation of various template MIPs. They are mainly used to exemplify the invention and do not restrict the scope of invention.

TABLE 1
Amino Acid Sequence of Example Peptides

| | Peptides | Amino Acid Sequence |
|---|---|---|
| Example1 and 2 | Oxytocin | 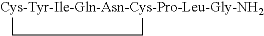 |
| Example 3 | Vasopressin | 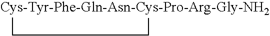 |
| Example 4 | Angiotension II | Asp-Arg-Val-Tyr-Ile-His-Pro-Phe |
| Example 5 | Bradykinin | Arg-Pro-Pro-Gly-Phe-Ser-Pro-Phe- |
| | | Arg |
| Example 6 | 15-mer peptide | Thr-Glu-Leu-Arg-Tyr-Ser-Trp-Lys- |
| | | Thr-Trp-Gly-Lys-Ala-Lys-Met |

### EXAMPLE 1

### Template Oxytocin (Preparation A)

### Step 1)

The QCM disks were immersed in a 10 μM solution of (Acr-L-Cys-NHBn)₂ in HPLC-grade acetonitrile for 16 hrs, then rinsed exhaustively with acetonitrile and dried under vacuum.

### Step 2)

A solution of acrylic acid (55 μmol), acrylamide (55 μmol), N-benzylacrylamide (110 μmol), and oxytocin (3 μmol) were mixed in 0.3 ml of acetonitrile water (1:1).

### Step 3)

After depositing 4 μl of the aforementioned aliquot on top of the (Acr-L-Cys-NHBn)₂ treated QCM disk, the chip was placed horizontally into a 20 ml vial containing acetonitrile (3 ml). The vial was capped and irradiated with UV-light at 350 nm for 6 hrs.

### Step 4)

The polymer, which was formed as a thin film on the electrode surface, was washed with 20 mM phosphate buffer (pH=3-4) to remove 70 to 80% of the oxytocin template. This was followed by a wash with acetonitrile and then placed in a drybox.

FIG. 3 illustrates the process of above steps. The thickness of the polymer films was measured as 92±15 nm on a surface profiler (Dekatak³-ST) from Veeco Inc.

The QCM frequency shifted −750±44 Hz after coating with (Acr-L-Cys-NHBn)₂ and shifted lower to −3400±800 Hz after polymerization. It shifted back 30±50 Hz after the removal of the template.

### EXAMPLE 2

### Template Oxytocin (Preparation B)

Repeated procedures of Example 1. However, in step (2), the amount of N-benzylacrylamide was reduced to 55 μmol.

### EXAMPLE 3

### Template Vasopressin

Repeated procedures of Example 1. However, in step (2), oxytocin was replaced by 3 μmol of vasopressin.

### EXAMPLE 4

### Comparative Template Angiotension II

Repeated procedures of Example 1. However, in step (2), oxytocin was replaced by 3 μmol of angiotension II.

### EXAMPLE 5

### Comparative Template Bradykinin

Repeated procedures of Example 1. However, in step (2), oxytocin was replaced by 3 μmol of bradykinin.

### EXAMPLE 6

### Comparative Template 15-mer Peptide

Repeated procedures of Example 1. However, in step (2), oxytocin was replaced by 3 μmol of a 15-mer peptide.

### (3) Evaluation of MIP-Grafted QCMs

A suitable means of analysis comprises a flow injection system made of a HPLC pump (Model L7110, Hitachi, flow rate=0.1 ml min⁻¹), home-built flow cell, sample injection valve (Model 1106, OMNIFIT), home-built oscillation circuit (including oscillator and frequency counter) and a personal computer. The system is filled with phosphate buffered saline (PBS, pH=7) solution.

Two O-rings were used to secure the polymer coated QCM onto the flow-cell. One side of the QCM disk was immersed in system buffer. Binding tests were performed on template and non-template peptide containing solutions, by injecting 100 μl aqueous solution of test samples and their frequency changes measured.

The results of oxytocin-imprinted QCM and vasopressin-imprinted QCM are shown in the figures below. As shown in FIGS. 4 and 5, the adsorption of related non-template peptides was not observed until the concentration of these peptides reached 1 ng/ml. The frequency shifts of three other peptides, angiotensin II, bradykinin, and the 15-mer peptide were compared in the same concentration. No trace was detected at 1 ng/ml. However, nonspecific adsorption of these peptides began to be visible when the concentration reached the level of 1 μg/ml.

The results of angiotensin II-imprinted QCM, bradykinin-imprinted QCM, and 15-mer peptide-imprinted QCM are shown in the figures below. As shown in FIGS. 6, 7 and 8, there was selectivity for the template peptide. Similarly, nonspecific adsorption of other non-template peptides can only be detected at higher concentration of the substrate.

To clearly demonstrate the binding abilities of MIPs, Bmax is set as the maximum frequency shift observed and B is the frequency shift obtained at the indicated concentration of peptide. FIGS. 9 and 10 show the binding effects of oxytocin-imprinted QCM and vasopressin-imprinted QCM, respectively. Thus, K_{d} values were calculated from the slope of curves. The best oxytocin MIP's K_{d} value was about 1.1*10⁻⁸ M (FIG. 9). The best vasopressin MIP's K_{d} value was about 2.0*10⁻⁸ M (FIG. 10). In general, MIP demonstrated a marked 10˜100 times enhancement in K_{d} value toward template-peptide than their nonspecific adsorptions to nontemplate-peptide.

The efficiency of the SAM procedure using affinity enhancer (Acr-L-Cys-NHBn)₂ for generating chiral surface with high affinity toward template molecules is evident. The presented chiral surface-imprinting technique fabricated on a QCM sensor is an ideal in situ analytical system for detecting peptides. It provides a convenient assay for quantitatively recognizing peptides at microscale.

1. A quartz crystal microbalance sensor comprising a metal surface, wherein said metal surface is first coated with a chiral monomer used as an affinity enhancer, a crosslinker and a photoinitiator; and a molecularly imprinted polymer film on said coated metal surface to detect at least one peptide in a solution.

2. The sensor as claimed in claim 1, wherein said chiral monomer is selected from a group comprising bisacryloyl-L-cystine-bis-benzylamide, bisacryloyl-L-cystine-bis-anilide, bismethacryl-L-cystine-bis-benzylamide, bismethacryl-L-cystine-bis-anilide, bisacryloyl-L-homocystine-bis-benzylamide, bisacryloyl-L-homocystine-bis-anilide, bismethacryl-L-homocystine-bis-benzylamide, bismethacryl-L-homocystine-bis-anilide, bisacryloyl-D-cystine-bis-benzylamide, bisacryloyl-D-cystine-bis-anilide, bismethacryl-D-cystine-bis-benzylamide, bismethacryl-D-cystine-bis-anilide, bisacryloyl-D-homocystine-bis-benzylamide, bisacryloyl-D-homocystine-bis-anilide, bismethacryl-D-homocystine-bis-benzylamide or bismethacryl-D-homocystine-bis-anilide.

3. The sensor as claimed in claim 1, wherein said chiral monomer is bisacryloyl-L-cystine-bis-benzylamide or bisacryloyl-D-cystine-bis-benzylamide or bismethacryl-L-cystine-bis-benzylamide or bismethacryl-D-cystine-bis-benzylamide.

4. The sensor as claimed in claim 1, wherein said chiral monomer is bisacryloyl-L-cystine-bis-benzylamide or bismethacryl-L-cystine-bis-benzylamide.

5. The sensor as claimed in claim 1, wherein said molecularly imprinted polymer is prepared by polymerization with template molecules wherein template molecules are disulfide bonded cyclic peptides.

6. The sensor as claimed in claim 1, wherein said molecularly imprinted polymer is prepared by polymerization with template molecules wherein template molecules are disulfide bonded cyclic peptides containing functional groups selected from cysteine, cystine, phenol, benzene, imidazole, guanidinium, alcohol, disulfide, thiol, or amide.

7. The sensor as claimed in claim 1, wherein said molecularly imprinted polymer is prepared by polymerization with template molecules wherein template molecules are oxytocin or vasopressin.

8. The sensor as claimed in claim 1, wherein said molecularly imprinted polymer is prepared by polymerization with template molecules wherein template molecules are angiotensin II, bradykinin, or a 15-mer peptide (Thr-Glu-Leu-Arg-Tyr-Ser-Trp-Lys-Thr-Trp-Gly-Lys-Ala-Lys-Met).

9. A quartz crystal microbalance sensor comprising a metal surface, wherein said metal surface is first coated with a chiral monomer comprised of bisacryloyl-L-cystine-bis-benzylamide or bismethacryl-L-cystine-bis-benzylamide or bisacryloyl-D-cystine-bis-benzylamide or bismethacryl-D-cystine-bis-benzylamide; and a molecularly imprinted polymer film on said coated metal surface to detect at least one peptide in a solution.

10. The sensor as claimed in claim 9, wherein said molecularly imprinted polymer is prepared by polymerization with template molecules wherein template molecules are disulfide bonded cyclic peptides.

11. The sensor as claimed in claim 9, wherein said molecularly imprinted polymer is prepared by polymerization with template molecules wherein template molecules are disulfide bonded cyclic peptides containing functional groups selected from cysteine, cystine, phenol, benzene, imidazole, guanidinium, alcohol, disulfide, thiol, or amide.

12. The sensor as claimed in claim 9, wherein said molecularly imprinted polymer is prepared by polymerization with template molecules wherein template molecules are oxytocin or vasopressin.

13. The sensor as claimed in claim 9, wherein said molecularly imprinted polymer is prepared by polymerization with template molecules wherein template molecules are angiotensin II, bradykinin, or a 15-mer peptide (Thr-Glu-Leu-Arg-Tyr-Ser-Trp-Lys-Thr-Trp-Gly-Lys-Ala-Lys-Met).

14. A quartz crystal microbalance sensor comprising a metal surface, wherein said metal surface is first coated with a chiral monomer comprised of bisacryloyl-L-cystine-bis-benzylamide or bismethacryl-L-cystine-bis-benzylamide; and a molecularly imprinted polymer film on said coated metal surface to detect at least one peptide in a solution.

15. The sensor as claimed in claim 14, wherein said molecularly imprinted polymer is prepared by polymerization with template molecules wherein template molecules are disulfide bonded cyclic peptides.

16. The sensor as claimed in claim 14, wherein said molecularly imprinted polymer is prepared by polymerization with template molecules wherein template molecules are disulfide bonded cyclic peptides containing functional groups selected from cysteine, cystine, phenol, benzene, imidazole, guanidinium, alcohol, disulfide, thiol, or amide.

17. The sensor as claimed in claim 14, wherein said molecularly imprinted polymer is prepared by polymerization with template molecules wherein template molecules are oxytocin or vasopressin.

18. The sensor as claimed in claim 14, wherein said molecularly imprinted polymer is prepared by polymerization with template molecules wherein template molecules are angiotensin II, bradykinin, or a 15-mer peptide (Thr-Glu-Leu-Arg-Tyr-Ser-Trp-Lys-Thr-Trp-Gly-Lys-Ala-Lys-Met).
